(19) United States
(12) Patent Application Publication
(10) Pub. No.: US 2024/0165009 A1
(43) Pub. Date: May 23, 2024

(54) HAIR STYLING AND HAIR CARE COMPOSITIONS COMPRISING A LEGUMINOUS-PLANT STARCH

(71) Applicant: ROQUETTE FRERES, Lestrem (FR)

(72) Inventors: Léon MENTINK, Lille (FR); Géraldine LOUVET-POMMIER, Lachelle (FR); Camille LACORE, Montmorency (FR); Caroline SORIN, Lille (FR)

(21) Appl. No.: 18/551,532

(22) PCT Filed: Mar. 17, 2022
(86) PCT No.: PCT/EP2022/025111

§ 371 Date: Sep. 20, 2023

(30) Foreign Application Priority Data
Mar. 25, 2021 (FR) 2103042

(51) Int. Cl. A61K 8/73; A61K 8/06; A61Q 5/00; A61Q 5/06

(52) U.S. Cl. CPC A61K 8/732; A61K 8/062; A61Q 5/004; A61Q 5/06

(57) ABSTRACT

The present invention relates to the field of cosmetic compositions, in particular hair compositions, for styling, shaping the hair, in particular shaping the hair in curls, and also the thermal protection of the hair. The invention also relates to the use of a leguminous-plant starch for shaping hair curls and also for preventing, eliminating or reducing thermal or chemical stress of hair.

### TECHNICAL FIELD

The present invention relates to the field of cosmetic compositions, in particular hair compositions, for styling, shaping the hair, in particular shaping the hair in curls, and also the thermal protection of the hair. The invention also relates to the use of a leguminous-plant starch for shaping hair curls and also for preventing, eliminating or reducing thermal or chemical stress of hair.

### PRIOR ART

People who have the curly hair may wish to obtain a better definition of their curls, a better regularity of the tightness or index of these curls, as well as curls which “bounce back” and are flexible.

Compositions have been proposed in the prior art to maintain hair curls while giving them good definition and regularity. Typically these compositions comprise associations of one or more fatty esters with one or more fixing polymers, thickening polymers and liquid fatty substances.

In addition, hair curls, in the same way as all types of hair, can be damaged by repeated drying and styling. Thermo-protective products to be applied to the hair prior to drying have been developed to provide a barrier to heat.

However, the compositions proposed in the prior art, whether to hold the curls, define them or to protect the hair from thermal stress, generally comprise thickeners which make the hair heavier, thickening them, stiffening them and thus leading to a “cardboard effect”, making the hair fixed and dull.

It is therefore necessary to be able to develop compositions which make it possible to define hair curls without making them heavier, and which can also protect hair from thermal stress generated by heating appliances like hair dryers or straightening or curling irons.

Moreover, there is a particularly strong demand for consumers for more natural products and a need to turn to alternative ingredients, which are friendlier to both hair and the environment.

It is thus necessary to identify compounds of natural origin and to develop cosmetic compositions for styling and care, allowing the shaping of the hair curls, without any “cardboard effect”, and which can also protect the hair from thermal stress generated by heating appliances.

### SUMMARY OF THE INVENTION

It is to the Applicant's credit to have demonstrated that modified leguminous-plant starches, preferentially pea starch, advantageously allow the shaping of hair curls, and preferentially makes it possible to give definition to hair curls.

Advantageously, the leguminous-plant starch according to the invention allows better definition of curls, better regularity of the spacing of the curl, and better movement, even in humid conditions. These effects are advantageously obtained without creating a “cardboard” effect. The hair remains light and natural.

According to a first aspect, the object of the present invention is the use of a modified leguminous-plant starch to increase the retention of hair curls and/or to maintain and preferentially increase the longevity of the compressive strength of the curl and/or to maintain and preferentially increase the longevity of the compressive stiffness of the curl.

Advantageously, the leguminous-plant starch according to the invention makes it possible to increase the retention of curls as well as to increase the longevity of the retention of the curl and its stiffness without any cardboard effect and while retaining a certain flexibility to the touch.

The Applicant has also demonstrated that the leguminous-plant starch according to the invention makes it possible to prevent and protect the hair from attacks induced by thermal and/or chemical stress. It has indeed been demonstrated that the leguminous-plant starch makes it possible to reduce the oxidation of the hair proteins on the cuticle and in the cortex of the hair. The hair is thus protected not only on the surface but also inside the capillary fiber.

Thus, and according to a second aspect, the present invention relates to the non-therapeutic use of a modified leguminous-plant starch to prevent, eliminate or reduce hair stress.

According to a third aspect, the present invention relates to specific cosmetic compositions.

These compositions advantageously make it possible to define and maintain hair curls and to protect hair from the stress generated not only by heating appliances, but also by the sun or chemicals, which could perm, decolorize, or uncurl the hair.

Thus, and according to a fourth aspect, the present invention relates to a method for shaping hair curls and/or for preventing or reducing hair stress, comprising a step of applying to said hair curls a cosmetic composition according to the invention.

The present invention also relates, according to a fifth aspect, to the use of the cosmetic compositions according to the invention for shaping hair curls and/or for preventing, eliminating or reducing hair stress.

### BRIEF DESCRIPTION OF THE DRAWINGS

FIG. 1 is an epifluorescence microscope image associated with densitometric analysis of the images of a sagittal view of a hair in order to quantify the oxidation of the hair in the cuticle and the cortex, on three groups of locks, a control group, a positive control group and a group treated with an aqueous solution of pea starch Beauté by Roquette® ST 720 (“BBR ST720 treated”).

FIG. 2 is an epifluorescence microscope image associated with densitometric analysis of the images of a longitudinal view of a hair in order to quantify the oxidation on the surface of the hair over a portion of its length, on three groups of locks, a control group, a positive control group and a group treated with an aqueous solution of pea starch Beauté by Roquette® ST 720 (“BBR ST720 treated”).

### DISCLOSURE OF THE INVENTION

Thus, the present invention relates to the non-therapeutic use of a modified leguminous-plant starch to increase the retention of hair curls and/or to maintain and preferentially increase the longevity of the compressive strength of the curl and/or to maintain and preferentially increase the longevity of the compressive stiffness of the curl. The longevity of the compressive strength or of the compressive stiffness corresponds to the resistance of the curl subjected to repeatedly applied mechanical forces.

Modified Leguminous-Plant Starch

Within the meaning of the present invention, “leguminous plant” means any plant belonging to the families of the cesalpiniaceae, mimosaceae or papilionaceae, and particularly any plant belonging to the family of the papilionaceae, for example pea, bean, broad bean, faba bean, lentil or lupin.

According to a preferred embodiment, the leguminous starch is a pea starch.

The leguminous starch according to the invention is obtained from starch with an amylose content of 25% to 45%, preferably from 30% to 45%, and preferably from 35% to 40%, where the percentages are expressed in dry weight relative to the dry weight of leguminous starch, and determined before any subsequent treatment such as hydrolysis and/or alkylation of said starch.

For the purposes of the present application, “modified” means that the starch has undergone at least one modification chosen from the thermal or chemical modifications of its macromolecular structure, or from the chemical modifications of substitution of the hydroxyls carried by the starch by other chemical groups. According to one embodiment, the modified leguminous-plant starch is chosen from acetylated leguminous-plant starches, octenyl succinate or hydroxyalkyl.

Thus, according to a preferred embodiment, the starch according to the invention is a hydrolyzed and hydroxyalkylated, preferably hydroxypropylated starch.

“Hydroxypropylated starch” is understood to mean, for the purpose of the present invention, starch substituted by hydroxypropyl groups by any technique known to the person skilled in the art, for example by etherification reaction with propylene oxide, in particular presenting a hydroxypropyl group content between 0.1 and 20% by dry weight relative to the dry weight of hydroxypropylated starch, preferably between 1 and 10%, more preferably between 5 and 9%, and in particular close to 7%. In particular, this content is determined by proton Nuclear Magnetic Resonance spectrometry, in particular according to the EN ISO 11543:2002 F standard.

“Hydrolyzed starch” is understood to mean, for the purpose of the present invention, a starch which has undergone a hydrolysis operation, i.e. an operation to reduce its mean molecular mass. The person skilled in the art knows how to obtain such starches, for example by chemical treatments such as oxidation and acid treatments, or also by enzymatic treatments. The person skilled in the art will naturally adjust the fluidification level of the starch based on the viscosity desired.

Hydrolyzed and alkylated leguminous-plant starch according to the invention may also comprise one or more other physical and/or chemical modifications, provided that said modifications do not interfere with the desired properties of said starch. An example of chemical modification particularly is cross-linking.

According to a particular embodiment, hydrolyzed and alkylated starch according to the invention may further have other modifications, and may for example have undergone physical treatments, particularly selected from the known gelatinization, pre-gelatinization, extrusion, atomization or drying operations, microwave or ultrasonic processing, plasticizing or granulation treatment operations.

In particular, starch after alkylation and hydrolysis will preferably be non-granular.

According to one embodiment, starch according to the invention is characterized in that it is rendered soluble. It can be made soluble by any technique known to the person skilled in the art, including by heat and/or mechanical treatment, for example by cooking in aqueous media, possibly followed by a drying step when obtaining a powder product is preferred. The operation to make the starch soluble may occur before the addition of the starch into the cosmetic composition, in an undifferentiated manner before or after the alkylation and/or hydrolysis of the starch, or after its addition into the cosmetic composition, for example by cooking the cosmetic composition at the time of its implementation.

The modified leguminous-plant starch, preferentially hydrolyzed and alkylated according to the invention has a preferred mean molecular weight of 1 to 5,000 kDa, preferably 10 to 3,000 kDa, more preferentially 20 to 2,000 kDa, and even more preferably 100 to 1,800 kDa. For example, the molecular weight may be about 1,500 kDa. The mean molecular weight is determined by HPSEC-MALLS (high-performance size exclusion chromatography coupled in-line with multi-angle laser light scattering detection).

Hydrolyzed and hydroxypropylated pea starches are commercially available and are available from the Applicant under the trademark Beauté by Roquette® ST 720.

Shaping Hair Curls

Various methods known to the person skilled in the art are available to measure the efficacy of the leguminous-plant starch according to the invention, for shaping hair curls and giving them definition. These properties are:
the retention of the curls, which is sub-divided into the retention of the length of the curls and the retention of the spacing of the curls;
the compression of the curls;
the bending of the curls.

Curl Retention

A method commonly used and known to the person skilled in the art is measuring curl retention, usually in “curl length”, but also in “curl tightness”. To characterize curl retention, two parameters are measured:
the length of the curls,
the tightness of the curls, also called the curl index, which is defined as the mean value of the irregular spiral winding that the curls describe.

The length of the curls must not or substantially be increased, and the tightness of the curls must be maintained, in order to conclude that the leguminous-plant starch can keep human hair curly.

These parameters are measured under so-called normal conditions, that is 70% relative humidity and 20° C., and in tropical conditions, that is 90% relative humidity and 35° C., for a period of 7 hours.

Typically, these parameters are measured by suspending hair locks, previously styled on a curler, on a sectioned, millimeter-graded grid placed in a humidity- and temperature-controlled atmosphere. The lengths to be monitored are then measured by reading the millimeter-graded grid over time.

The retention of the curls in terms of length of a curled lock is calculated according to the formula:

% length retention=[Length of the lock before styling−Length of the styled lock at time t]/[Length of the lock before styling−Length of the styled lock right after drying(t0)]×100

The length of the lock before styling (i.e. before curling) is the length between the base of the lock and its end, standardized at 26 cm for the locks of our tests. The length of the styled (that is to say, curled) lock at time to, respectively at time t, is the length between the base of the lock and its end just after having placed the styled (that is to say, curled) lock in the controlled-atmosphere chamber, respectively after a duration t in said chamber.

The retention of the curl tightness for a curled lock is calculated according to the formula:

% tightness retention=[Total length of the tightnesses of a styled lock at time to−Average of tightnesses at time t]/[Total length of the tightnesses of a styled lock at time to−Average of tightnesses at time t0]×100

The total length of the styled lock tightnesses at time to is the distance between the base of the styled lock and the end of the styled lock (generally 17 cm in our curl tests). The average of the tightnesses at time to, respectively at time t, is the average of the values of the tightnesses of each well formed on the styled lock just after having placed the styled lock in the controlled-atmosphere chamber, respectively after a duration t in said chamber.

In terms of retention of the length of the curls and of tightness retention of the curls, the leguminous-plant starch according to the invention advantageously makes it possible to reduce the drop in the percentage of retention between the time to and the instant t=1 h, and also makes it possible to increase the average value of the plateau where the percentage of retention between 3 and 7 hours after styling stabilizes.

The leguminous-plant starch according to the invention advantageously makes it possible to increase the retention of the length of the curls, which results in a reduction in the initial decrease in the length of the curls, and by an increase in the value at which this length typically stabilizes 3 hours after styling.

Under temperate relative humidity and temperature (that is to say, 70% RH and 20° C.), the leguminous-plant starch according to the invention makes it possible to maintain the initial reduction in the retention of the curls in terms of length at a value of less than 35%, preferentially less than 30%, and most preferentially less than 25%. The leguminous-plant starch according to the invention makes it possible to stabilize the retention of the curls in terms of length at a value greater than or equal to 55%, preferentially greater than or equal to 60%, more preferentially greater than or equal to 65%, and most preferentially greater than or equal to 70%.

Under tropical relative humidity and temperature (that is to say, 90% RH and 35° C.), the leguminous-plant starch according to the invention makes it possible to maintain the initial reduction in the retention of the curls in terms of length at a value of less than 75%, preferentially less than 60%, and most preferentially less than 45%. The leguminous-plant starch according to the invention makes it possible to stabilize the retention of the curls in terms of length at a value greater than or equal to 25%, preferentially greater than or equal to 35%, more preferentially greater than or equal to 40%.

The leguminous-plant starch according to the invention advantageously makes it possible to increase the retention of the tightness of the curls, which results in a reduction in the initial decrease in the tightness of the curls, and by an increase in the value at which the tightness typically stabilizes 3 hours after styling.

Under temperate relative humidity and temperature (that is to say, 70% RH and 20° C.), the leguminous-plant starch according to the invention makes it possible to maintain the initial reduction in the retention of the curls in terms of tightness (or index) at a value less than or equal to 4%, preferentially less than or equal to 2%. The leguminous-plant starch according to the invention makes it possible to stabilize the retention of the curls in terms of tightness (or index) at a value greater than or equal to 94%, preferentially greater than or equal to 96%, more preferentially greater than or equal to 97%.

Under tropical relative humidity and temperature (that is to say, 90% RH and 35° C.), the leguminous-plant starch according to the invention makes it possible to maintain the initial reduction in the retention of the curls in terms of tightness (or index) at a value of less than 30%, preferentially less than 20%, and most preferentially less than 10%. The leguminous-plant starch according to the invention makes it possible to stabilize the retention of the curls in terms of tightness (or index) at a value greater than or equal to 60%, preferentially greater than or equal to 65%, more preferentially greater than or equal to 80%.

According to one embodiment, the leguminous-plant starch according to the invention advantageously makes it possible to maintain a retention of the curls in terms of length of at least 60%, preferentially of at least 65%, and most preferentially of at least 70%, for a duration of at least 1 hour, preferentially at least 2 hours, preferentially at least 3 hours, preferentially for at least 5 hours, and most preferentially for at least 7 hours.

According to one embodiment, the leguminous-plant starch according to the invention advantageously makes it possible to maintain a retention of the tightness of the curls of at least 94%, preferentially of at least 97%, for a duration of at least 1 hour, preferentially at least 2 hours, preferentially at least 3 hours, preferentially for at least 5 hours, and most preferentially for at least 7 hours.

The leguminous-plant starch therefore has styling properties for the retention of the curls, whether in terms of the retention of the length of the curls or retention of the tightness of the curls.

Thus, the present invention relates to the use of a modified leguminous-plant starch to increase the retention of the hair curls, and preferentially to increase the retention of the length of the curls and/or the retention of the tightness of the curls.

Curl Compression

The measurement of the compression of the curls makes it possible to evaluate the mechanical strength of a curl under the effect of a mechanical stress, in particular a compressive stress, that is a force exerted perpendicularly to a tangent to the curl toward the center of the curl. This typically involves measuring the force necessary to deform the curl. Two parameters will be measured:
the longevity of the compressive strength;
the longevity of the compressive stiffness.

It is possible to characterize the longevity of the strength and the longevity of the compressive stiffness of a hair curl by measuring the value of the force, and its gradient, required to compress said curl during a set number of compression-relaxation cycles, typically 10 cycles. Processing software then calculates:
the longevity of the compressive strength: this is the complement to 100% of the absolute value of the relative variation of the maximum force before breakage of the film of styling product between the tenth cycle and the first cycle relative to the first cycle, expressed as a %; the greater the value, the greater the longevity of the compressive strength,
the longevity of the compressive stiffness: this is the complement to 100% of the absolute value of the relative variation of the maximum force before breakage of the film of styling product between the tenth cycle and the first cycle relative to the first cycle, expressed as a %; the greater the value, the greater the longevity of the compressive stiffness.

Typically, these parameters can be measured by an apparatus of the brand Dia-Stron®, the “MTT175”, by following the “curl compression” protocol.

Advantageously, the leguminous-plant starch according to the invention ensures the longevity of the compressive strength of the curl as well as the longevity of the compressive stiffness of the curl.

The leguminous-plant starch according to the invention advantageously makes it possible to achieve a longevity of the compressive strength of the curls greater than 85%, preferentially greater than 90%. The leguminous-plant starch according to the invention advantageously enables a longevity of the compressive stiffness of the curls greater than 75%, preferentially greater than 85%.

Thus, the present invention relates to the use of a modified leguminous-plant starch to maintain and preferentially increase the longevity of the compressive strength of the curl and/or to maintain and preferentially increase the longevity of the compressive stiffness of the curl.

According to a preferred embodiment, the starch according to the invention makes it possible to increase the retention of the hair curls and to maintain and preferentially increase the longevity of the compressive strength of the curl and to maintain and preferentially increase the longevity of the compressive stiffness of the curl.

The retention of the curls and the longevity of the compressive strength and stiffness of the curl must ideally be obtained without any “cardboard effect”. More reasonably, it is the obtaining of a reduced or moderate cardboard effect which is generally achievable. Indeed, the curl must remain fairly flexible and must not be too stiffened by the leguminous-plant starch. The cardboard effect, or the flexibility, are evaluated according to the three-point bending method below.

Three-Point Bending

Three-point bending measurement makes it possible to evaluate the flexibility of a lock of flat styled hair. A lock of styled hair in the form of a flat strip is subjected to bending at its center, with its ends placed on immobile supports. This method is used to evaluate the binding, long-term strength and “cardboard effect”. The apparatus measures the force required to reversibly fold the tress, or to break the film definitively, and calculates four parameters as a result:
the maximum flexural strength: this is the maximum force to be applied to break the film of styling product on the lock,
the maximum flexural stiffness: this is the maximum of the force gradient to break the film of styling product,
the longevity of the flexural strength: this is the complement to 100% of the relative variation of the maximum force before breakage of the film of styling product between the tenth cycle and the first cycle relative to the first cycle, expressed as a %; the greater the value, the greater the longevity of the strength,
the longevity of the flexural stiffness: this is the complement to 100% of the relative variation of the maximum force before breakage of the film between the tenth cycle and the first cycle relative to the first cycle, expressed as a %; the greater the value, the greater the longevity of the stiffness.

Typically, these parameters can be measured according to the “3-point bend” protocol on the Dia-Stron® MTT175.

The modified leguminous-plant starch according to the invention makes it possible to increase the maximum flexural strength of a styling composition by at least 10%, preferentially by at least 20%, more preferentially by at least 30%, and most preferentially by at least 40%, relative to the maximum flexural strength of the same styling composition without the leguminous-plant starch according to the invention. The modified leguminous-plant starch according to the invention makes it possible to decrease the maximum flexural stiffness of a styling composition by at least 10%, preferentially by at least 20%, and most preferentially by at least 30%, relative to the maximum flexural stiffness of the same styling composition without the leguminous-plant starch according to the invention.

According to a preferred embodiment, the modified leguminous-plant starch according to the invention makes it possible to increase the maximum flexural strength, while reducing the maximum flexural stiffness: it therefore enables stronger curls, while reducing the cardboard effect.

The modified leguminous-plant starch according to the invention also makes it possible to increase the longevity of the flexural strength of a curl, advantageously to a value greater than or equal to 92%, preferentially greater than or equal to 95%, and most preferentially greater than or equal to 98%. The modified leguminous-plant starch according to the invention also makes it possible to increase the longevity of the flexural stiffness of a curl.

Thus, by combining all the preceding effects, the modified leguminous-plant starch according to the invention allows better retention of the curls, improves their attachment, has an effect over the long term, with a moderate cardboard effect, or even without a cardboard effect.

Thermal Protection

The present invention also relates to the non-therapeutic use of a modified leguminous-plant starch to prevent, eliminate or reduce hair stress.

The stress may be a thermal stress and/or a chemical stress.

Typically, thermal stress is induced by regular or irregular drying of the hair and/or the use of a straightening iron or any heating apparatus, but also by UV rays during exposure to the sun.

Typically, the chemical stress is induced by the application of oxidizing products, for example those contained in hair dyes or colorings, full or partial hair color removal products, curling products, whether intended to do so permanently or defrizz. Chemical stress is also induced by frequent exposure to chlorine.

The thermal or chemical stress will lead to an oxidation of the hair proteins via a carbonylation reaction. The hair will then be weakened and damaged.

It has also been demonstrated that the leguminous-plant starch according to the invention makes it possible to prevent and protect the hair from attacks induced by thermal and/or chemical stress. It has indeed been demonstrated that the leguminous-plant starch makes it possible to reduce the oxidation of the hair proteins in the cuticle and in the cortex of the hair. The hair is thus protected not only on the surface but also inside the capillary fiber.

Thus, according to one embodiment, the present invention also relates to the non-therapeutic use of a modified leguminous-plant starch to prevent, eliminate or reduce the oxidation of the hair induced by thermal stress.

According to one embodiment, the present invention also relates to the non-therapeutic use of a modified leguminous-plant starch to prevent, eliminate or reduce the oxidation of the hair induced by chemical stress.

The modified leguminous-plant starch according to the invention makes it possible to protect the hair from thermal and/or chemical stress.

Forms of Administration

According to one embodiment, the present invention also relates to a cosmetic composition comprising a leguminous-plant starch modified in a physiologically acceptable medium.

The physiologically acceptable medium is preferably cosmetically or dermatologically acceptable. “Physiologically acceptable medium” or “cosmetically acceptable” or “dermatologically acceptable” is understood to mean a medium which does not present deleterious side effects and in particular does not produce redness, inflammation, heating, tightness or tingling unacceptable for a user of cosmetic or dermatological products. The medium is thus compatible with the keratinous materials of human beings.

The cosmetic composition comprises 0.1% to 30%, preferably 0.5% to 20%, and particularly 0.75% to 10% by weight of modified leguminous-plant starch. The percentages by weight being expressed in relation to the total weight of the cosmetic composition.

The cosmetic composition according to the invention may be in any pharmaceutical form used in the field of hair and normally used for application to hair such as aqueous, hydroalcoholic or oily solutions, lotion- or serum-type dispersions or solutions, milk-type emulsions of liquid or semi-liquid consistency, obtained by dispersion of a fatty phase in an aqueous phase (O/W) or vice-versa (W/O), cream-type emulsions, aqueous gels, microemulsions, microcapsules, microparticles, or ionic and/or nonionic vesicular dispersions.

These compositions are prepared according to the usual methods known to the person skilled in the art.

According to one embodiment, the composition according to the invention may be in the form of emulsions, that is to say compositions comprising an aqueous phase and an oil phase dispersed in each other, for example water-in-oil (W/O) or oil-in-water (O/W) or multiple (W/O/W or O/W/O) emulsions.

According to one embodiment, the composition is in the form of a water-in-oil emulsion.

Water-in-oil (W/O) emulsions comprise an aqueous phase dispersed in an oily phase. These emulsions comprise an oily continuous phase. According to one preferred embodiment, the composition is in the form of an oil-in-water emulsion. These emulsions comprise an oily phase dispersed in an aqueous phase. These emulsions comprise an aqueous continuous phase.

Among the preparations in the form of oil-in-water or water-in-oil emulsions or in the form of a microemulsion suitable for application to the scalp and the hair, one may mention preparations for a “rinse-off” application: shampoos, conditioners, hair masks, serums, foams, balms, creams, sprays, detanglers, dyes or colorings, hair color removal or defrizzing products, or “leave-on” preparations intended to be applied to the hair without rinsing, known as emulsions, gels, creams, aqueous gels, sprays, and serums.

Aqueous Phase

The cosmetic composition according to the invention comprises an aqueous phase comprising water and optionally one or more water-miscible organic solvents.

According to one embodiment, the water-in-oil emulsion according to the invention comprises from 40% to 95% by weight of water, preferably from 50% to 90%, preferably from 55% to 85%, preferably from 60% to 80%, by weight of water relative to the total weight of the composition.

According to one embodiment, the oil-in-water emulsion according to the invention comprises from 10% to 90% by weight of oil, preferably from 25% to 75%, preferably from 40% to 70%, preferably from 45% to 65%, by weight of water relative to the total weight of the composition.

The composition may comprise, in the aqueous phase, at least one water-soluble solvent.

The term “water-soluble solvent” refers to a compound that is liquid at room temperature and miscible with water (miscibility in water greater than 50% by weight at 25° C. and atmospheric pressure).

Water-soluble solvents that can be used in the compositions according to the invention can be volatile.

Among the water-soluble solvents that can be used in the compositions in accordance with the invention, mention may be made of monoalcohols having from 1 to 5 carbon atoms such as ethanol and isopropanol, C₃-C₄ ketones and C₂-C₄ aldehydes.

Thus, according to one preferred embodiment, the composition according to the invention comprising at least one monoalcohol having from 1 to 5 carbon atoms, preferably ethanol.

Typically, the monoalcohol having 1 to 5 carbon atoms is present at a content ranging from 0.1 to 25% by weight, preferably from 0.5 to 10% by weight, preferably from 1 to 5% by weight, relative to the total weight of the composition.

Humectants

The emulsion according to the invention may further contain at least one humectant. The humectant may be chosen from polyols and/or esters of fatty acids and of polyethylene glycol.

“Polyols” is understood to mean any molecule having in its structure at least two free hydroxy (—OH) groups. These polyols are preferably liquid at room temperature (25° C.).

Typically, the polyol will be selected from maltitol, mannitol, xylitol, erythritol, sorbitol, isosorbide, glycerol or glycerin, glucose, sucrose, polydextrose, hydrogenated glucose syrups, dextrins, maltodextrins, glucose syrups, and mixtures thereof.

By way of illustration, mention will be made of the product Beauté by Roquette® PO 070 or PO 071 (INCI: Sorbitol), PO 160 (sorbitol powder) PO 260 (mannitol), PO 370 (xylitol), PO 500, sold by ROQUETTE, Glycerin (INCI: glycerin) sold by the company COOPER, propylene glycol (INCI: propylene glycol) from the company COOPER, Butylene Glycol (INCI: 1,3-BUTANEDIOL).

Among the esters of fatty acids and of polyethylene glycol, one may mention the product sold under the name Glucamate SSE-20 (INCI: PEG-20 METHYL GLUCOSE SESQUISTEARATE) by the company LUBRIZOL ADVANCED MATERIALS, Inc.

The composition comprises from 0.1% to 25% by weight of polyols, preferably from 0.5% to 20% by weight, and even more preferably from 1% to 10% by weight of polyols, relative to the total weight of the composition.

Oily Phase

The cosmetic composition according to the invention comprises at least one oil.

For the purposes of the present invention, “oil” is understood to mean a compound which is liquid at room temperature (25° C.) and which, when it is introduced at an amount of at least 1% by weight into the water at 25° C., is not at all soluble in the water, or soluble to an extent of less than 10% by weight, relative to the weight of oil introduced into the water.

According to one embodiment, the oil will be chosen from volatile oils, non-volatile oils and mixtures thereof.

The liquid fatty phase advantageously comprises one or more nonvolatile oils which afford an emollient effect on the skin.

The term “non-volatile oil” means an oil remaining on the keratin materials at room temperature and atmospheric pressure for at least several hours and especially having a vapor pressure of less than 10-3 mmHg (0.13 Pa).

Among non-volatile oils, mention may be made of fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, triacetin, tricaprin, caprylic/capric acid triglycerides, coco caprate and caprylate mixture, benzoates of C12 to C15 alcohols, glycol esters such as butylene glycol cocoate, glyceryl triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, vegetable oils such as avocado oil, camellia oil, hazelnut oil, tsubaki oil, cashew nut oil, argan oil, soybean oil, grape seed oil, sesame oil, corn oil, wheatgerm oil, rapeseed oil, sunflower oil, cottonseed oil, jojoba oil, peanut oil, macadamia oil, sweet almond oil, olive oil and mixtures thereof.

These non-volatile oils may also be hydrocarbon or silicone type oils, such as paraffin oils, squalane, petroleum jelly, dimethyl siloxanes and mixtures thereof.

According to one embodiment, the non-volatile oil is chosen from stearic acid, jojoba oil (INCI: Simmondsia chinensis Seed Oil), grape seed oil (INCI: Vitis vinifera seed oil), macadamia oil (INCI: Macadamia ternifolia seed oil), refined oleic sunflower oil (Helianthus annuus seed oil), the mixture of caprate and coco caprylate such as the product Miglyol Coco 810 (INCI: Coco-Caprylate/Caprate), sweet almond oil (INCI: Prunus Amygdalus Dulcis Oil), sesame oil (INCI: Sesamum indicum seed oil).

Mention will be made, among fatty esters, of the product DUB VCI 10 (INCI: Isodecyl Neopentanoate) from the company STEARINERIE DUBOIS.

According to one embodiment, the non-volatile oil is present at a content ranging from 10% to 90% by weight, preferably from 25% to 75% by weight, preferably from 40% to 70% by weight, preferably 45% to 65% by weight, relative to the total weight of the composition.

The liquid fatty phase may also comprise volatile oils. Volatile oil is intended to mean an oil which is capable of evaporating from the skin in less than one hour at room temperature and atmospheric pressure. The volatile oils may for example be selected from silicone oils or short-chain fatty acid triglycerides in order to reduce the greasy feel.

According to one embodiment, the volatile oil is present at a content ranging from 10% to 90% by weight, preferably from 25% to 75% by weight, preferably from 40% to 70% by weight, preferably 45% to 65% by weight, relative to the total weight of the composition.

The fatty or oily phase of the composition according to the invention may further advantageously comprise at least one fatty phase structuring agent such as a wax.

Wax

“Wax” is intended to mean a fatty substance having reversible liquid-solid state change and having a melting point of greater than 30° C. and generally less than 90° C., which is liquid under the preparation conditions of the composition and which has anisotropic crystalline organization in the solid state. The waxes used according to the invention may consist of polar or apolar waxes or a mixture of these two. “Apolar” is intended to mean a wax containing only carbon, hydrogen and/or phosphorus atoms, and in particular a hydrocarbon.

The polar waxes may be selected from animal waxes, vegetable waxes, and synthetic or silicone waxes containing polar groups such as esters. Mention may thus be made of carnauba wax, candelilla wax, beeswax (Cera alba), Chinese insect wax (Ericerus pela), Japan wax, sumac wax, montan wax, triesters of C8-C20 acids and glycerin, such as glyceryl tribehenate, acetylated glycol stearate, sold particularly by VEVY under the trade name CETACENE, and mixtures thereof. These waxes may in particular be used in predispersed form in an oil, as is the case of the mixture of candelilla wax and jojoba seed oil.

Mention will also be made of butters such as shea butter, cocoa butter, camellia butter.

According to one embodiment, the wax is present at a content ranging from 0.5 to 50% by weight, preferably from 1 to 25% by weight, preferably from 3 to 10% by weight, relative to the total weight of the composition.

Gelling Agent

According to one embodiment, the composition according to the invention comprises at least one gelling agent.

A gelling agent refers to a compound which, in the presence of a solvent, creates more or less strong intermacromolecular bonds, thus inducing a three-dimensional network which fixes said solvent.

The gelling agent is chosen from hydrophilic organic gelling agents, and/or lipophilic organic gelling agents, and/or amphiphilic organic gelling agents, and/or mineral gelling agents.

The gelling agent makes it possible to confer viscosity on the continuous phase, that is to say to adjust the viscosity of the continuous phase to a value ranging from 100 mPa·s to 20,000 mPa·s.

The organic gelling agent can be chosen from polymers of synthetic origin or of plant origin, preferentially of plant origin, chemically modified or not. It can thus be chosen from gums derived from plants such as gum arabic, konjac gum, guar gum or derivatives thereof; gums extracted from algae such as alginates; gums derived from microbial fermentation such as xanthans, for example, the product Keltrol CG (INCI: xanthan gum), sold by the company CP KELCO or the product Xanthan Gum FNCS-PC (INCI: xanthan gum) sold by the company JUNGBUNZLAUER INTERNATIONAL AG, mannans, scleroglucans or derivatives thereof; cellulose and derivatives thereof such as carboxymethylcellulose or hydroxyethylcellulose; starch and derivatives thereof such as modified starches, notably acetylated, carboxymethylated, octenylsuccinates or hydroxypropylated; synthetic polymers such as polyacrylic acids or carbomers, and mixtures thereof. Typically, a carboxymethylated potato starch may be used, such as Beauté by Roquette® ST 118 sold by the company ROQUETTE.

Among the carbomers, mention will for example be made of the product Carbopol Ultrez 30 (INCI: carbomer) from the company LUBRIZOL ADVANCED MATERIALS, Inc. or CARBOPOL ETD 2050 POLYMER (INCI: carbomer).

Mention will also be made of mixtures of gum arabic and xanthan gum such as the product Solagum AX (INCI: Acacia Senegal gum (and) Xanthan gum), sold by the company SEPPIC, mixtures of starch and cellulose derivatives such as the product Beauté by ROQUETTE® DS112 (INCI: Starch acetate (and) Hydroxyethylcellulose (and) Xanthan gum), sold by the company ROQUETTE.

The mineral gelling agent may be selected from magnesium and/or aluminum silicates. An example of such a mineral gelling agent is Veegum® Pure from Vanderbilt Minerals LLC, which is a magnesium aluminum silicate.

Surfactant

According to one embodiment, the composition according to the invention comprises at least one surfactant selected from cationic surfactants, anionic surfactants, nonionic surfactants, zwitterionic or amphoteric surfactants, and mixtures thereof. The role of said at least one surfactant may be to emulsify the oil in water or water in the oil, or to clean and/or to condition the keratin fibers.

Cationic Surfactant

Cationic surfactants in particular comprise the salts of primary, secondary or tertiary, optionally polyoxyalkylenated fatty amines, quaternary ammonium salts, and mixtures thereof.

Among quaternary ammonium salts, tetraalkylammonium chlorides are preferred, on the one hand, such as dialkyldimethylammonium or alkyltrimethylammonium chlorides wherein the alkyl group has approximately 12 to 22 carbon atoms, in particular behenyltrimethylammonium, distearyldimethylammonium, cetyltrimethylammonium, benzyldimethylstearylammonium chlorides or, on the other hand, distearoylethylhydroxyethylmethylammonium methosulfate, dipalmitoylethylhydroxlammonium methosulfate or distearoylethylhydroxyethylammonium methosulfate, palmitylamidopropyltrimethylammonium chloride or stearamidopropyldimethyl-(myristyl acetate)-ammonium chloride, marketed under the name CERAPHYL® 70 by the company VAN DYK; quaternary ammonium salts of imidazoline, marketed for example under the name REWOQUAT® W 75 by the company REWO.

Among quaternary di- or triammonium salts, mention will be made, for example, of Finquat CT-P available from FINETEX (Quaternium 89), Finquat CT available from FINETEX (Quaternium 75).

Among quaternary ammonium salts containing one or more ester functions, mention will be made of chloride or methylsulfate of diacyloxyethyldimethylammonium, of diacyloxy ethyl hydroxyethyl methyl ammonium, of monoacyloxyethyldihydroxyethyl methyl ammonium, of triacyloxyethyl methyl ammonium, of monoacyloxy ethylhydroxy ethyldimethylammonium, and mixtures thereof. The acyl groups preferably have 14 to 18 carbon atoms and more particularly come from a vegetable oil such as palm oil or sunflower oil. When the compound contains several acyl groups, they may be identical or different. These products are obtained, for example, by direct esterification of triethanolamine, triisopropanolamine, alkyldiethanolamine or alkyldiisopropanolamine optionally oxyalkylenated on fatty acids or on mixtures of fatty acids of vegetable or animal origin, or by transesterification of methyl esters thereof. This esterification is followed by quaternization using an alkylating agent such as an alkyl halide, preferably methyl or ethyl, a dialkyl sulfate, preferably methyl or ethyl, methyl methanesulfonate, methyl para-toluenesulfonate, glycol chlorohydrin or glycerol chlorohydrin. Such compounds are for example sold under the names DEHYQUART® by the company HENKEL, STEPANQUAT® by the company STEPAN, NOXAMUM® by the company CECA, REWOQUAT® WE 18 by the company REWOWITCO.

The composition may contain, for example, a mixture of quaternary ammonium mono-, di- and tri-ester salts with a majority by weight of diester salts. It is also possible to use ammonium salts containing at least one ester function described in U.S. Pat. Nos. 4,874,554 and 4,137,180. Use may be made of behenoyl-hydroxypropyltrimethylammonium chloride provided by KAO under the name Quatarmin BTC 131.

Preferably, the ammonium salts containing at least one ester function contain two ester functions. Among the cationic surfactants that can be present in the composition, it is more particularly preferred to choose salts of cetyltrimethylammonium, of behenyltrimethyl ammonium, of dipalmitoylethylhydroxyethylmethylammonium, and mixtures thereof, and more particularly behenyltrimethylammonium chloride, cetyltrimethylammonium chloride, dipalmitoylethylhydroxyethylammonium methosulfate, and mixtures thereof.

The composition according to the invention may comprise one or more cationic surfactants in a total content ranging from 0.01 to 15% by weight, preferably from 0.1 to 10% by weight, and more preferentially from 0.5 to 5% by weight, better still from 1 to 3% by weight relative to the total weight of the composition

Anionic Surfactant

The anionic surfactants are chosen from carboxylic anionic surfactants, sulfate-based anionic surfactants, sulfonate surfactants, phosphate anionic surfactants, and mixtures thereof, preferably from sulfonate anionic surfactants, carboxylic anionic surfactants, and mixtures thereof.

“Anionic surfactant” is intended to mean a surfactant which only comprises anionic groups as ionic or ionizable groups. In the present description, an entity is described as being “anionic” when it has at least one permanent negative charge or when it can be ionized into a negatively charged entity, under the conditions of use of the composition of the invention (medium, pH for example) and not comprising any cationic charge.

“Sulfate-based anionic surfactant” means an anionic surfactant comprising at least one sulfate function (—OSO₃H or —OSO₃), and may optionally further comprise one or more other functions derived from acids, such as carboxylic acid or carboxylate functions (—COOH or —COO—), sulfonate functions (—SO₃H or —SO₃) and/or phosphate functions. By way of example, alkyl sulfates, alkyl ether sulfates, alkylamidoether sulfates, alkylarylpolyethersulfates, monoglyceride sulfates, and also the salts of these compounds, are sulfate-based anionic surfactants. The alkyl groups of these compounds cited by way of example comprise 6 to 30 carbon atoms, and the aryl group denotes a phenyl or benzyl group. These compounds cited by way of example may be polyoxyalkylenated, in particular polyoxyethylenated, and comprise from 1 to 50 ethylene oxide units. Among the anionic surfactants of alkyl sulfate type, mention may be made of alkyl sulfate salts and mixtures thereof, in particular in the form of alkali metal (N or K) or alkaline earth metal (for example Mg) or ammonium, amine or amino alcohol salts, and mixtures thereof. The sulfate anionic surfactant may be sodium cocosulfate which is the sodium salt of the coconut alcohol ester which generally has the formula: ROSO3Na, where R represents the alkyl groups derived from coconut oil. The INCI name is sodium cocosulfate. A preferred sodium cocosulfate according to the present invention is that sold by Rhodia under the name MACKOL™ CAS-100N.

“Non-sulfate-based anionic surfactant” is intended to mean a surfactant which does not fall within the definition of “sulfate-based anionic surfactant” as defined above.

It is also intended, according to the invention, that:
the carboxylic anionic surfactants comprise at least one carboxylic or carboxylate function (—COOH or —COO⁻), but does not comprise a sulfonic or sulfonate function (—SO₃H or —SO₃⁻), nor sulfate function (—OSO₃H or —OSO₃⁻);
the sulfonate anionic surfactants comprise at least one sulfonic or sulfonate function (—SO₃H or —SO₃), and may optionally further comprise one or more carboxylic or carboxylate functions (—COOH or —COO⁻) and/or phosphates, but do not comprise a sulfate function (—OSO₃H or —OSO₃⁻).
the phosphate anionic surfactants comprise at least one phosphoric or phosphate function (—OPO₃H₂ or —OPO₃²⁻), but do not comprise a carboxylic or carboxylate function (—COOH or —COO—), nor a sulfonic or sulfonate function (—SO₃H or —SO₃), nor a sulfate function (—OSO₃H or —OSO₃).

In other words:
an anionic surfactant comprising at least one sulfate function (—OSO₃H or —OSO₃⁻), and at least one carboxylic or carboxylate function (—COOH or —COO⁻), is considered, within the meaning of the invention and unless otherwise indicated, as a sulfate-based anionic surfactant;
an anionic surfactant comprising at least one sulfate function (—OSO₃H or —OSO₃), and at least one sulfonic or sulfonate function (—SO₃H or —SO₃), is considered, within the meaning of the invention and unless otherwise indicated, as a sulfate-based anionic surfactant;
an anionic surfactant comprising at least one sulfonic or sulfonate function (—SO₃H or —SO₃), and at least one carboxylic or carboxylate function (—COOH or —COO⁻), is considered, within the meaning of the invention and unless otherwise indicated, as a non-sulfated sulfonate anionic surfactant; ·an anionic surfactant comprising at least one sulfate function (—OSO₃H or —OSO₃), and at least one sulfonic or sulfonate function (—SO₃H or —SO₃), and at least one carboxylic or carboxylate function (—COOH or —COO⁻), is considered, within the meaning of the invention and unless otherwise indicated, as a sulfate-based anionic surfactant.

The carboxylic anionic surfactants can be chosen from the following compounds: acylglycinates, acyllactylates, acylsarcosinates, acylglutamates, alkyl-D-galactoside-uronic acids, alkyl ether carboxylic acids, alkyl(aryl)ether carboxylic acids, alkyl(amido)ether carboxylic acids; and the salts of these compounds. The alkyl and/or acyl groups of these compounds comprise from 6 to 30 carbon atoms, more preferentially from 8 to 28, more preferentially still from 10 to 24, even better still from 12 to 22 carbon atoms. The aryl group preferably denotes a phenyl or benzyl group. These compounds may be polyoxyalkylenated, in particular polyoxyethylenated, and then preferably comprise from 1 to 50 ethylene oxide units, better still from 2 to 10 ethylene oxide units. It is also possible to use C6-C24 alkyl monoesters and polyglycoside-polycarboxylic acids such as C6-C24 alkyl polyglycoside citrates, C6-C24 alkyl polyglycoside tartrates, and salts thereof.

According to one embodiment, the carboxylic anionic surfactants are chosen from:
C6-C24, or even C12-C20 acylglutamates, such as stearoylglutamates, and in particular sodium stearoyl glutamate or disodium cocoylglutamates, in particular sodium or disodium cocoyl glutamate;
acylsarcosinates, in particular C6-C24 or even C12-C20 acylsarcosinates, such as cocoyl sarcosinates and in particular sodium cocoyl sarcosinate, lauroyl sarcosinates and in particular sodium lauroyl sarcosinate, palmitoylsarcosinates, and in particular sodium palmitoylsarcosinate;
acyllactylates, in particular C12-C28 or even C14-C24 acyllactylates, such as behenoyllactylates, and in particular sodium behenolyl lactylate, (iso)stearoyl lactylates, and in particular sodium (iso)stearoyl lactylate;
acylglycinates, in particular C6-C24 or C12-C20 acylglycinates, such as cocoyl glycinates and in particular sodium cocoyl glycinate;
alkyl(C6-C30)ether carboxylic acids, in particular alkyl(C6-C24)ether carboxylic acids;
alkyl(C6-C30)arylether carboxylic acids, in particular alkyl(C6-C24)arylether carboxylic acids;
alkyl(C6-C30)amidoether carboxylic acids, in particular alkyl(C6-C24)amidoether carboxylic acids;
and mixtures thereof; and in particular in the form of alkali or alkaline earth metal salts, ammonium, or aminoalcohol.

The anionic sulfonate surfactants can be selected from the following compounds: alkylsulfonates, alkylamidesulfonates, alkylarylsulfonates, C6-C24 alkyl polyglycoside sulfosuccinates, alpha-olefin sulfonates, kerosene sulfonates, alkylsulfosuccinates, alkyl ether sulfosuccinates, alkylamidesulfosuccinates, alkylsulfoacetates, N-acyltaurates, acyl isethionates; alkyl sulfolaurates; and salts of these compounds; the alkyl groups of these compounds containing from 6 to 30 carbon atoms, in particular from 12 to 28, even better from 14 to 24, or even from 16 to 22, carbon atoms; the aryl group preferably designating a phenyl or benzyl group; these compounds may be polyoxyalkylenated, in particular polyoxyethylenated and in such case preferably containing from 1 to 50 ethylene oxide units, better still from 2 to 10 ethylene oxide units.

According to one embodiment, the anionic sulfonate surfactants are chosen from: C6-C24, in particular C12-C20 alkyl sulfosuccinates, especially lauryl sulfosuccinates; C6-C24, in particular C12-C20 alkyl ether sulfosuccinates; (C6-C24)acyl isethionates, preferably (C12-C18)acyl isethionates; alpha-olefin sulfonates; and mixtures thereof; and in particular in the form of alkali or alkaline-earth metal, ammonium or aminoalcohol salts.

According to one embodiment, the anionic surfactants may be chosen from C6-C24 alkyl monoesters and polyglycoside-dicarboxylic acids such as alkyl glucoside citrates, alkyl polyglycosidet tartrates and alkyl polyglycosidesulfosuccinates, alkylsulfosuccinamates, acyl isethionates and N-acyltaurates, the alkyl or acyl group of all these compounds preferably comprising from 12 to 20 carbon atoms. Another group of anionic surfactants that can be used in the compositions of the present invention is that of acyllactylates, the acyl group of which comprises from 8 to 20 carbon atoms. Furthermore, other examples include alkyl-D-galactosideuronic acids and their salts, as well as polyoxyalkylenated (C6-C24)alkyl ether carboxylic acids, polyoxyalkylenated (C6-C24)alkyl(C6-C24 aryl)ether carboxylic acids, polyoxyalkylenated (C6-C24 alkyl)amidoether carboxylic acids and their salts, in particular those with 2 to 50 ethylene oxide units, and mixtures thereof.

The phosphate anionic surfactants are chosen from: C6-C24 alkyl phosphates, in particular C12-C20 alkyl phosphates; C6-C24 alkyl ether phosphates, in particular C12-C20 alkyl ether phosphates; and mixtures thereof.

Nonionic Surfactants

According to one embodiment, the nonionic surfactant is chosen from:
alcohols, alpha-diols and alkyl(C1-C20)phenols, these compounds being polyethoxylated and/or polypropoxylated and/or polyglycerolated, the number of ethylene oxide and/or propylene oxide groups being able to range from 1 to 100, and the number of glycerol groups possibly ranging from 2 to 30; or else these compounds comprising at least one fatty chain comprising from 8 to 40 carbon atoms, in particular from 16 to 30 carbon atoms; in particular, alcohols comprising at least one saturated or non-linear or branched, oxyethylenated, C8-C40 alkyl chain comprising from 1 to 100 mol of ethylene oxide, preferably from 2 to 50, more particularly from 2 to 40 mol of ethylene oxide and comprising one or two fatty chains;
ethylene oxide and propylene oxide condensates on fatty alcohols;
ethers of fatty alcohols and of polyethylene glycol;
polyethoxylated fatty amides preferably having from 2 to 30 ethylene oxide units, polyglycerolated fatty amides comprising, on average from 1 to 5 glycerol groups and in particular from 1.5 to 4;
non-ethoxylated sorbitan fatty acid esters, ethoxylated or polyethoxylated fatty acid esters preferably having from 2 to 40 ethylene oxide units;
fatty acid esters of sucrose, fatty acid and glycerol esters, fatty acid and sucrose esters or polyesters, fatty acid and polyethylene glycol esters;
polyoxyalkylenated fatty acid esters, preferably polyoxyethylenated, having from 2 to 150 moles of ethylene oxide, including oxyethylenated vegetable oils;
N-(alkyl en C6-C24) glucamine derivatives,
amine oxides such as (C10-C14 alkyl)amine oxides or N—(C10-C14 acyl)-aminopropylmorpholine;
polyether-modified polysiloxanes;
and mixtures thereof.

Examples of fatty acid esters include Citrol GMS 40 (INCI: Glyceryl stearate), sold by the company CRODA, or Imwitor 960K (INCI: Glyceryl stearate), sold by the company BIESTERFELD, Imwitor 372P (INCI: Glyceryl stearate citrate), sold by the company BIESTERFELD, Glucate SS (INCI: Methyl Glucose Sesquistearate), sold by the company LUBRIZOLADVANCED MATERIALS, Inc, plurol stearique (Polyglyceryl-6 Distearate), Natragem E145 (INCI: Polyglyceryl-4 Laurate/Succinate (and) Aqua), sold by the company CRODA, Span 60 (INCI: sorbitan stearate), sold by the company SIGMA ALDRICH. Among the sorbitan esters, mention will for example be made of the product Span 20. Among the fatty alcohols, mention will for example be made of the product sold under the name Promulgen D (INCI: Cetearyl alcohol (and) Ceteareth-20).

According to another embodiment, the nonionic surfactant is selected from alkyl(poly)glycosides, which are represented by the following general formula:

R₁0−(R₂0)ₜ-(G)ᵥ
wherein:
R₁ represents a linear or branched alkyl or alkenyl radical comprising 6 to 24 carbon atoms, or 6 to 18 carbon atoms, or 6 to 12 carbon atoms; or an alkylphenyl radical, the linear or branched alkyl radical of which comprises 6 to 24 carbon atoms, or 6 to 18 carbon atoms, or 6 to 12 carbon atoms,
R₂ represents an alkylene radical comprising 2 to 4 carbon atoms,
G represents a sugar unit having 5 to 6 carbon atoms,
t denotes a value ranging from 0 to 10, preferably from 0 to 4,
v denotes a value ranging from 1 to 15, preferably from 1 to 4.

Preferably, the alkyl(poly)glycoside surfactants are compounds of the formula described above wherein:
R₁ denotes a saturated or unsaturated, linear or branched alkyl radical comprising 6 to 18 carbon atoms, or 6 to 12 carbon atoms,
R₂ represents an alkylene radical comprising 2 to 4 carbon atoms,
t denotes a value ranging from 0 to 3, preferably equal to 0,
G denotes glucose, fructose or galactose, preferably glucose;
the degree of polymerization, that is the value of v, which may range from 1 to 15, preferably from 1 to 4; the average degree of polymerization being more particularly between 1 and 2.

The glucosidic bonds between sugar units are generally of type 1-6 or 1-4, preferably of type 1-4. Preferably, the alkyl(poly)glycoside surfactant is an alkyl(poly)glucoside surfactant, that is an alkyl(poly)glycoside surfactant where G is a glucose. The alkyl C6/C16-(poly)glucosides 1,4, C6/C12-(poly)glucosides 1,4, and in particular and in particular decyl glucosides 1,4, dodecylglucosides, the hetpoglucosides, caprylyl glucosides, capryl glucosides and capryl/capryl glucosides, are most particularly preferred.

Among the commercial products, mention may be made of the products sold by the company COGNIS under the names PLANTAREN® (600 CS/U, 1200 and 2000) or PLANTACARE® (818, 1200 and 2000); products sold by SEPPIC under the names ORAMIX CG 110 and ORAMIX® NS 100, Montanov 68 (INCI: CETEARYL ALCOHOL (AND) CETEARYL GLUCOSIDE), sold by the company SEPPIC, Montanov L (INCI: C14-C22 Alcohols & C12-20 Alkyl Glucoside), sold by the company SEPPIC, Montanov 202 (INCI: Arachidyl Alcohol (and) Behenyl Alcohol (and) Arachidyl Glucoside); products sold by the company BASF under the name LUTENSOL GD 70; and products sold by the company CHEM Y under the name AG10 LK. The nonionic surfactant may also be chosen from alkyl(poly)glycosides, preferentially alkyl(poly)glucosides, with an HLB greater than or equal to 10, or greater than or equal to 12, or greater than or equal to 14. The alkyl(poly)glycosides, preferentially the alkyl(poly)glycosides, may also be combined with fatty alcohols.

Emulsifier Systems

The surfactant may be advantageously selected from oil-in-water emulsifying systems consisting of a cyclodextrin and a water-in-oil emulsifier of natural origin, such as the oil-in-water emulsifying system sold by Roquette Frères under the name Beauté by Roquette® DS146.

It will also be possible to use emulsifiers based on plant-based gums and modified starches such as the product Beauté by ROQUETTE® DS 421, sold by the company ROQUETTE.

Amphoteric or Zwitterionic Surfactant

The amphoteric or zwitterionic surfactants may be chosen from secondary or tertiary or quaternary aliphatic amine derivatives, in particular betaine surfactants, and lecithins, hydrogenated lecithins.

Among the secondary, tertiary or quaternary aliphatic amine derivatives, it is possible to choose from those whose aliphatic group is a linear or branched chain comprising from 8 to 22 carbon atoms and containing at least one anionic group such as, for example, a carboxylate, sulfonate, sulfate, phosphate or phosphonate group. Mention may also be made of: alkyl(C8-C20)betaines, for example cocobetaine, sulfobetaines, alkyl(C8-C20)sulfobetaines, alkyl(C8-C20)amidoalkyl(C3-C8)betaines, for example cocamidopropylbetaine; or alkyl(C8-C20)amidoalkyl(C6-C8)sulfobetaines.

Among betaine surfactants, it is possible in particular to choose from:
alkyl betaines, in particular lauryl betaine, such as for example the product GENAGEN KB® from CLARIANT, or cocobetaine, for example the product Dehyton AB 30® from BASF or TEGO Betain AB 1214 of Evonik Goldschmidt GmbH;
N-alkylamido betaines and derivatives thereof, in particular cocamidopropyl betaine (for example, LEBON 2000 HG® from SANYO or EMPIGEN BB® from ALBRIGHT ET WILSON), lauramidopropyl betaine (for example REWOTERIC AMB12PR from WITCO), or N-di-sodium N-carboxyethoxyethyl N-cocoylamidoethyl aminoacetate (INCI name: disodium cocoamphodiacetate; for example MIRANOL C2M CONCENTRE NP® from RHODIA CHIMIE);
sultaines, such as cocoyl amidopropyl hydroxy-sulfobetaine (for example CROSULTAINE C-500 from CRODA);
and mixtures thereof

Binder

According to one embodiment, the composition further comprises a binder.

Preferentially, the solid compositions according to the invention comprise at least one binder.

The term “binder” means compounds conferring increased cohesion of the cosmetic composition. This cohesion can be adjusted as a function of the amount and the chemical affinity of the binder relative to the ingredients of the cosmetic composition.

Typically, the binder will be chosen from caprylic/capric acid triglicerides such as the product Labrafac CC (INCI: capric/caprylic triglicerides) or Cetyl Dimethicone (INCI).

The binder will be present in the composition according to the invention at a content ranging from 0.5% to 20%, preferably from 1 to 15%, preferably from 5 to 12%, by weight relative to the total weight of the composition.

Advantageously, the binders will also have emollient properties.

Fatty Phase Viscosifying Agent

According to one embodiment, the composition according to the invention may further comprise at least one fatty phase viscosifying agent, that is, a lipophilic or amphiphilic viscosifying agent chosen from fatty alcohols.

Lipophilic or amphiphilic viscosifying agent is understood to mean a molecule or macromolecule capable of increasing the viscosity of an oil without substantially changing the physical or chemical properties thereof.

Among the fatty alcohols, mention will be made of Lanette 16 (INCI: cetyl alcohol), Lanette O (INCI: cetearyl Alcohol).

Dyestuff

The composition according to the invention may further comprise at least one dyestuff selected from water-soluble or liposoluble dyes, fillers with the effect of coloring and/or opacifying the composition and/or coloring the keratinous materials, preferentially the skin, such as pigments, nacres, lakes (water-soluble dyes adsorbed on an inert mineral support) and mixtures thereof. These dyestuffs may be optionally surface-treated by a hydrophobic agent such as silanes, silicones, fatty acid soaps, C9-15 fluoroalcohol phosphates, acrylate/dimethicone copolymers, C9-15 fluoroalcohol phosphate/silicon mixed copolymers, lecithins, carnauba wax, polyethylene, chitosan and optionally amino acids which could be acylated such as lauroyl lysine, disodium stearoyl glutamate and aluminum acyl glutamate, phytic acid.

“Pigments” means white or colored, mineral or organic particles intended to color and/or opacify the cosmetic composition.

Among the pigments, mention will be made of mineral or organic, natural or synthetic pigments. Examples of pigments are especially iron, titanium or zinc oxides, and also composite pigments and goniochromatic, pearlescent, interference, photochromic or thermochromic pigments, with this list being non-limiting.

Among the pigments surface-treated with lecithins are UNIPURE WHITE LC981 HLC (INCI: CI 77891 (and) Hydrogenated Lecithin, UNIPURE YELLOW LC182 HLC (INCI: CI 77492 (and) Hydrogenated Lecithin), UNIPURE BLACK LC989 HLC (INCI: CI 77499 (and) Hydrogenated Lecithin, UNIPURE RED LC381HLC (INCI: CI 77491 (and) Hydrogenated Lecithin) from the company Sensient Cosmetic Technologies.

Among the pigments surface-treated with phytic acid are Unipure White LC 985 PHY (INCI: CI 77891 (and) Phytic Acid (and) Sodium Hydroxide), Unipure Yellow LC 188 PHY (INCI: CI 77492 (and) Phytic Acid (and) Sodium Hydroxide), Unipure Red LC 388 PHY (INCI: CI 77491 (and) Phytic Acid (and) Sodium Hydroxide), Unipure Black LC 998 PHY(INCI: CI 77499 (and) Phytic Acid (and) Sodium Hydroxide), from the company Sensient Cosmetic Technologies.

“Nacres” is intended to mean any iridescent or non-iridescent colored particles which have a color effect by optical interference.

Among nacres, mention will be made of titanium mica covered with metal oxide, such as iron oxide or titanium oxide.

Fillers

The cosmetic composition according to the invention may further contain at least one filler. This term is intended to mean particles of any form (especially spherical or lamellar), mineral or organic, which are insoluble in the composition. Examples of fillers are talc, boron nitride, starch, polyamides, silicone resins, silicone elastomer powders and acrylic polymer powders, in particular powders of poly(methyl methacrylate) or powders of styrene acrylate copolymer (Sunsphere Powders from Dow).

Typically, it will be possible to use an aluminum starch octenylsuccinate (AOS), such as the product Beauté by Roquette® ST 012 sold by the company ROQUETTE, or, native starches, such as a native corn starch such as the product Beauté by Roquette® ST 005 or cyclodextrins such as the product Beauté by Roquette® CD 100.

Active Ingredients

The composition according to the invention may also comprise at least one cosmetic active ingredient.

Mention will be made, as cosmetic active ingredient or cosmetically active ingredient or cosmetically active substance, of an antioxidant agent, an anti-radical agent, a hydrating agent, an emollient, a vitamin pollution control agent, minerals, etc.

Among the vitamins, mention will be made of tocopherol, for example the product Covi-ox® T-70 C (INCI: tocopherol), sold by the company BASF, which can be used as an antioxidant.

Typically, the cosmetic active ingredient is present at a content ranging from 0.1% to 15% by weight, relative to the total weight of the composition, preferably from 0.5 to 10% by weight, and even more preferably from 1 to 5% by weight relative to the total weight of the composition.

Additives

The cosmetic compositions according to the invention may comprise other ingredients provided that they do not interfere with the desired properties of the composition. These other ingredients may for example, preservatives such as sodium benzoate (INCI: Sodium benzoate) like the product Microcare NB, sold by the company Thor, or potassium sorbate (INCI: Potassium sorbate), such as the product Microcare KS, also sold by the company Thor, pH adjusters such as citric acid, soda or arginine, fragrances, sunscreens, chelating agents, antimicrobial agents such as antimicrobial liquid compositions, for example an antimicrobial liquid composition comprising an essential oil chosen from the essential oils of species of the Cymbopogon type, such as essential oils of citronella, and essential oils of West Indian lemongrass, East Indian lemongrass, an alkyl(poly)glucoside, a gluconic acid and its salt, sodium gluconate.

Among the sunscreens, mention will be made, by way of illustration, of Solaveil XT 300 (INCI: Titanium Dioxide (and) Caprylic/Capric Triglyceride (and) Polyhydroxystearic Acid (and) Stearic Acid (and) Alumina), from CRODA, EUSOLEX 2292 (INCI: ETHYLHEXYL METHOXYCINNAMATE) or EUSOLEX T-2000 (INCI: CI 77891 & ALUMINA & SIMETHICONE) from the company Merck KGaA.

According to one embodiment, the emulsions according to the invention will be prepared with “bio-sourced” ingredients. “Bio-sourced” is understood to mean an ingredient whose dry mass in carbon atoms of renewable origin is at least 50% by weight of the total dry mass of carbon atoms.

The emulsions according to the invention will be prepared by any technique known to the person skilled in the art.

Cosmetic Compositions

According to one embodiment, the cosmetic composition that is the subject matter of the present application is an oil-in-water emulsion comprising, and preferentially consisting of:
at least one leguminous-plant starch,
at least one volatile oil,
at least one non-volatile oil,
at least one wax,
at least one surfactant,
water.

According to one embodiment, the cosmetic composition that is the subject matter of the present application is an oil-in-water emulsion comprising, and preferentially consisting of:
at least one leguminous-plant starch,
at least one volatile oil,
at least one non-volatile oil,
at least one wax,
at least one surfactant,
water,
at least one gelling agent,
at least one colorant.

According to one embodiment, the cosmetic composition that is the subject matter of the present application is an oil-in-water emulsion comprising, and preferentially consisting of:
at least one leguminous-plant starch,
at least one volatile oil,
at least one non-volatile oil,
at least one wax,
at least one surfactant,
water,
at least one gelling agent,
at least one binder,
at least one fatty phase viscosifying agent,
at least one colorant,
at least one filler.

According to one embodiment, the cosmetic composition that is the subject matter of the present application is an oil-in-water emulsion comprising, and preferentially consisting of:
at least one leguminous-plant starch,
at least one volatile oil,
at least one non-volatile oil,
at least one wax,
at least one surfactant,
water,
at least one gelling agent,
at least one binder,
at least one fatty phase viscosifying agent,
at least one colorant,
at least one filler,
at least one cosmetic active ingredient

Method for Shaping Hair Curls and/or Preventing, Removing or Reducing Thermal Stress of Hair

The present invention also relates to a method for shaping hair curls and/or for preventing, removing or reducing hair stress comprising a step of applying a composition according to the invention to the hair.

This application step may optionally be preceded by a step of washing the hair.

The application step may optionally be followed by a setting step, for example a setting time of 1 to 15 minutes, preferentially 2 to 5 minutes, then optionally followed by a rinsing step or not, for example with water, and/or a drying step. These embodiments will apply during the application of rinse-off preparations.

In the case of “leave-on” preparations, the hair is not rinsed after the step of applying the composition according to the invention. Typically, the composition will be applied to the hair at least 1 hour, and preferentially for the day in order to allow styling and definition of the curls. The application step will optionally be followed by a drying step.

The compositions according to the invention then make it possible to define the curls and to shape them, and protect the hair from thermal stress induced by heating appliances or the sun, but also chemical stress induced by oxidizing products.

Non-Therapeutic Use of Hair Styling Compositions

The present invention also relates to the non-therapeutic use of a cosmetic composition as described above, for shaping hair curls.

Typically, the cosmetic compositions according to the invention make it possible to increase the retention of hair curls and/or to maintain and preferentially to increase the longevity of the curl strength and/or to maintain and preferentially to increase the longevity of the curl stiffness.

The present invention also relates to the non-therapeutic use of a cosmetic composition according to the invention, to prevent, eliminate or reduce thermal stress of the hair.

Typically, the cosmetic compositions according to the invention make it possible to prevent, eliminate or reduce the oxidation of the hair induced by thermal stress.

### EXAMPLES

In the examples below, the ingredients listed in table 1 are used.

TABLE 1

| Ingredient | INCI | Supplier |
|---|---|---|
| Water | Aqua | — |
| Beauté by Roquette ® | Hydroxypropyl starch | Roquette |
| ST 720 | | |
| Beauté by Roquette ® | Starch Acetate (and) | Roquette |
| DS 112 | Hydroxyethyl Cellulose (and) | |
| | Xanthan Gum | |
| Beauté by Roquette ® | Hydrogenated Starch | Roquette |
| PO 455 | Hydrolysate | |
| Beauté by Roquette ® | Cyclodextrin (and) Sorbitol (and) | Roquette |
| DS 146 | Polyglyceryl-3 Diisostearate | |
| Beauté by Roquette ® | Sorbitol | Roquette |
| PO 070 | | |
| Kelcogel CG-HA | Gellan Gum | CP kelco |
| Montanov 68 | Cetearyl Alcohol & Cetearyl | SEPPIC |
| | Glucoside | |
| Lanette 16 | Cetyl Alcohol | BASF |
| Cegesoft VP | Olus Oil (and) Hydrogenated | BASF |
| | Vegetable Oil (and) Euphorbia | |
| | Cerifera (Candelilla) Wax | |
| Argan oil | Argania Spinosa Kernel Oil | DSM |
| Lipovol C-76 | Cocos Nucifera (Coconut) Oil | Vantage |
| Shea butter | Butyrospermum Parkii (Shea) | Cooper |
| | Butter | |
| Phenoxyethanol | Phenoxyethanol | Jan Dekker |
| Coconut oil | Cocos nucifera oil | VANTAGE |
| Sunflower oil | Helianthus annuus seed oil | VANTAGE |
| Alcohol Denat | Alcohol | — |
| Grapeseed oil | Vitis vinifera seed oil | — |
| IMWITOR ® 372 P | Glyceryl stearate citrate | IOI Oleo |
| | | GmbH |
| IMWITOR ® 900 K | Glyceryl stearate | IOI Oleo |
| | | GmbH |
| Shea butter | Butyrospermum Parkii (Shea) | Cooper |
| | Butter | |
| Microcare PE | Phenoxyethanol | Jan Dekker |

### Example 1: Curl Retention

Here, the ability of aqueous solutions of fixing agents, and cosmetic formulations in cream form, to retain/maintain curls, that is, retain/maintain the styling of curly human hair, is evaluated. The stability of two curl retention criteria is evaluated: the length of the curls, and the index (or tightness) of the curls. The stability of these criteria is evaluated by monitoring over a duration of 4 to 7 or 8 hours according to the tests, under controlled temperature and humidity conditions. Temperate conditions are 70% relative humidity and 20° C. Tropical conditions are 90% relative humidity and 35° C.

The retention of the curls in terms of length of a curled lock is calculated according to the formula:

% length retention=[Length of the lock before styling−Length of the styled lock at time t]/[Length of the lock before styling−Length of the styled lock right after drying(t0)]×100

The length of the lock before styling is the length between the base of the lock and its end, standardized at 26 cm for the locks of our tests. The length of the styled lock at time to, respectively at time t, is the length between the base of the lock and its end just after having placed the styled lock in the controlled-atmosphere chamber, respectively after a duration t in said chamber.

The retention of the curl tightness for a curled lock is calculated according to the formula:

% tightness retention=[Total length of the tightnesses of astyled lock at time to−Average of tightnesses at time t]/[Total length of the tightnesses of astyled lock at time to−Average of tightnesses at time t0]×100

The total length of the styled lock tightnesses at time to is the distance between the base of the styled lock and the end of the styled lock (generally 17 cm in our curl tests). The average of the tightnesses at time to, respectively at time t, is the average of the values of the tightnesses of each well formed on the styled lock just after having placed the styled lock in the controlled-atmosphere chamber, respectively after a duration t in said chamber.

The locks used for these tests are locks of standardized Caucasian hair at 0.8 g and 25 cm of length (EXTIFF-brand micro-ring natural extensions). Each lock is shaped into curls and evaluated for its curl retention according to the following protocol:

1/Washing a Lock:

A lock is impregnated with water by dipping it into a still water bath for 6 seconds. 0.4 g of sodium laureth sulfate (Texapon NSA SLES 28% DM) is then applied by spreading it all along the lock. The lock is rubbed ten times with zigzag movements (5 times on one side, and 5 times on the other side). The lock of hair is rinsed under water for 40 seconds (20 seconds on one side, 20 seconds on the other). The excess water is removed by wiping three times between two fingers. The lock is then left flat to dry for 12 hours.

2/Application of the Styling Product:

The lock is moistened under tap water for 5 seconds on each side. The excess water is removed by squeezing it out between two fingers. 0.3 g of styling product is applied to the lock by spreading over the entire length of the lock. The lock is rubbed ten times with zigzag movements (5 times on one side, and 5 times on the other side). The lock is carefully installed on the roller (describe the roller) and it is fixed appropriately.

3/Drying and Measurement:

The support is placed covered with the lock in an oven at 50° C. for 1.5 hours. After drying, the lock of the support is carefully removed, and it is attached to the milliliter-marked plate. The total length of the lock is then noted. The milliliter-marked plate is then placed where all the required locks were installed, in a climate oven at 20° C. and 70% relative humidity. The characteristic lengths are then measured every hour for at least 7 hours.

This starts by evaluating aqueous solutions according to the compositions of table 2.

TABLE 2

| | Solu- | Solu- | Solu- | Solu- | Solu- |
|---|---|---|---|---|---|
| | tion 1 | tion 2 | tion 3 | tion 4 | tion 5 |
| Water (% weight) | 100 | 99 | 95 | 99 | 95 |
| Polyvinylpyrrolidone | 0 | 1 | 5 | 0 | 0 |
| (% weight) | | | | | |
| Beauté by Roquette ® | 0 | 0 | 0 | 1 | 5 |
| ST 720 (% weight) | | | | | |

The protocol for evaluating the retention of the curls is applied under normal environment conditions (70% RH, 20° C.), and the results of tables 3 and 4 are obtained.

In table 3 below, the measurements of the retention of the length of the curls at 70% RH and 20° C. are listed.

TABLE 3

| | Length retention (%) | | | | |
|---|---|---|---|---|---|
| Time (hour) | Solution 1 | Solution 2 | Solution 3 | Solution 4 | Solution 5 |
| 0 | 100 | 100 | 100 | 100 | 100 |
| 1 | 42 | 49 | 62 | 71 | 80 |
| 2 | 37 | 44 | 58 | 67 | 78 |
| 3 | 34 | 38 | 56 | 63 | 74 |
| 4 | 34 | 38 | 54 | 61 | 74 |
| 5 | 34 | 38 | 53 | 59 | 74 |
| 6 | 37 | 38 | 52 | 51 | 73 |
| 7 | 34 | 38 | 52 | 65 | 76 |
| Initial | 58 | 51 | 38 | 29 | 20 |
| decrease | | | | | |
| Decrease | 8 | 11 | 10 | 6 | 4 |
| 1-7 hours | | | | | |
| Average | 35 | 38 | 53 | 62 | 74 |
| plateau | | | | | |
| 3-7 hours | | | | | |

It is observed that the retention of the length of the locks styled with the solutions 4 and 5 is much better than that of the locks styled with the polyvinylpyrrolidone, both regarding the initial decrease (that occurring in the first hour) and the decrease between 1 hour and 87 hours, as well as the value at which it stabilizes (that is, the average value of the plateau where the retention stabilizes).

Table 4 below shows the measurements relating to the retention of the tightness of the curls at 70% RH and 20° C.

TABLE 4

| | Tightness retention (%) | | | | |
|---|---|---|---|---|---|
| Time (hour) | Solution 1 | Solution 2 | Solution 3 | Solution 4 | Solution 5 |
| 0 | 100 | 100 | 100 | 100 | 100 |
| 1 | 88 | 92 | 97 | 96 | 98 |
| 2 | 188 | 88 | 96 | 95 | 197 |
| 3 | 86 | 87 | 95 | 94 | 197 |
| 4 | 186 | 86 | 94 | 94 | 97 |
| 5 | 86 | 86 | 96 | 94 | 97 |
| 6 | 85 | 86 | 92 | 93 | 97 |
| 7 | 85 | 86 | 92 | 93 | 97 |
| Initial | 12 | 8 | 3 | 4 | 2 |
| decrease | | | | | |
| Decrease | 3 | 6 | 5 | 3 | 1 |
| 1-7 hours | | | | | |
| Average | 86 | 86 | 93 | 94 | 97 |
| plateau | | | | | |
| 3-7 hours | | | | | |

It is observed that the curl tightness retention of the aqueous solutions 4 and 5 is equivalent to or greater than solutions 2 and 3. Solution 4 containing only 1% of styling agent gives a retention equivalent to solution 3 which contains 5 times more styling agent. The highest curl tightness retention is achieved with solution 5 containing 5% of Beauté by Roquette ST 720.

The pea starch Beauté by Roquette ST 720 therefore has better styling properties for curl retention than polyvinylpyrrolidone, in terms of both retention of the lock lengths, and retention of the curl tightnesses.

The retention of the curls is then evaluated with solutions 3 and 5 under tropical conditions (90% HR and 35° C.). The results of tables 4 and 5 are obtained.

Table 5 shows the measurements relating to the retention of the curls in terms of length and tightness at 90% HR and 35° C.

TABLE 5

| | Length retention (%) | | Tightness retention (%) | |
|---|---|---|---|---|
| Time (hour) | Solution 3 | Solution 5 | Solution 3 | Solution 5 |
| 0 | 100 | 100 | 100 | 100 |
| 1 | 27 | 57 | 73 | 93 |
| 2 | 25 | 47 | 71 | 90 |
| 3 | 25 | 42 | 71 | 88 |
| 4 | 23 | 40 | 70 | 85 |
| 5 | 23 | 40 | 66 | 84 |
| 6 | 21 | 38 | 66 | 82 |
| 7 | 21 | 37 | 63 | 82 |
| 8 | 21 | 37 | 62 | 80 |
| Initial | 73 | 43 | 27 | 7 |
| decrease | | | | |
| Decrease | 5 | 20 | 10 | 11 |
| 1-4 hours | | | | |
| Average | 23 | 39 | 67 | 84 |
| plateau | | | | |
| 2-4 hours | | | | |

It is observed that the retention of the curls under tropical conditions with a styling solution containing the BBR ST720 pea starch is better than with a styling solution containing polyvinylpyrrolidone, both for curl length retention and curl tightness retention.

A hairstyling cream of the leave-on type “Styling cream HC-010-018” according to the composition of table 6 is then evaluated.

TABLE 6

| | | Placebo | Styling cream with |
|---|---|---|---|
| | | styling | Beauté by |
| Phase | Ingredient | cream | Roquette ® ST 720 |
| JA1 | Water | q.s. 100 | q.s. 100 |
| A1 | Beauté by Roquette ® ST 720 | 0 | 6 |
| A2 | Beauté by Roquette ® DS 112 | 4 | 4 |
| B | Montanov 68 | 4 | 4 |
| B | Lanette 16 | 1 | 1 |
| B | Cegesoft VP | 5 | 5 |
| B | Argan oil | 6 | 6 |
| B | Lipovol C-76 | 3 | 3 |
| B | Shea butter | 3 | 3 |
| C | Phenoxyethanol | 1 | 1 |

The curl retention evaluation protocol is applied, and the results in table 7 are obtained.

TABLE 7

| | Length retention (%) | | Tightness retention (%) | |
|---|---|---|---|---|
| Time (hour) | placebo | BBR ST720 | Placebo | BBR ST720 |
| 0 | 100 | 100 | 100 | 100 |
| 1 | 52 | 65 | 90 | 95 |
| 2 | 46 | 56 | 89 | 92 |
| 3 | 40 | 54 | 188 | 90 |
| 4 | 40 | 54 | 88 | 90 |
| Initial | 48 | 35 | 10 | 5 |
| decrease | | | | |
| Decrease | 12 | 10 | 1 | 5 |
| 1-4 hours | | | | |
| Average | 42 | 55 | 89 | 91 |
| plateau | | | | |
| 2-4 hours | | | | |

The curl retention styling properties of the placebo cream are improved by the addition of the pea starch Beauté by Roquette® ST720. The initial decrease in the length of the lock length retention is reduced, and the length retention stabilizes at a higher value. Likewise, the tightness retention decreases by less initially, and stabilizes at a higher average value.

Other examples of cosmetic formulations comprising the pea starch Beauté by Roquette® ST 720 as a styling agent, and thus having better curl length and tightness retentions, are given below.

The formula “Curly spray HC-020-004” is presented in table 8.

TABLE 8

| Phase | Ingredient | % w/w |
|---|---|---|
| A1 | Water | q.s. 100 |
| A1 | Beauté by Roquette ® ST 720 | 5 |
| A1 | Beauté by Roquette ® PO 455 | 18 |
| A2 | Kelcogel CG-HA | 0.1 |
| A3 | Beauté by Roquette ® DS 146 | 3 |
| B | Coconut oil | 4 |
| B | Sunflower oil | 6 |
| C | Phenoxyethanol | 0.7 |

The formula “Styling gel HC-021-002” is presented in table 9.

TABLE 9

| Phase | Ingredient | % w/w |
|---|---|---|
| A1 | Water | q.s. 100 |
| A1 | Beauté by Roquette ® DS 112 | 5 |
| A1 | Beauté by Roquette ® PO 070 | 3 |
| A2 | Beauté by Roquette ® ST 720 | 7 |
| B | Alcohol Denat | 5 |
| B | Microcare PE | 1 |

The formula “Beach wave styler HC-008-005” is presented in table 10.

TABLE 10

| Phase | Ingredient | % w/w |
|---|---|---|
| A1 | Water | q.s. 100 |
| A1 | Beauté by Roquette ® DS 112 | 3 |
| A2 | Beauté by Roquette ® ST 720 | 5 |
| B | Vitis vinifera seed oil | 1 |
| B | Glyceryl stearate citrate | 0.5 |
| B | Glyceryl stearate | 0.5 |
| B | Butyrospermum Parkii (Shea) Butter | 0.5 |
| C | Microcare PE | 1 |

Thus, and advantageously, these compositions make it possible to increase the retention of the hair curls and make it possible in particular to increasing curl length retention and/or curl tightness retention.

### Example 2: Curl Compression

Here the retention of a curl is evaluated in terms of compression according to the “curl compression” measurement protocol of the “MTT175” apparatus from the manufacturer “Dia-Stron Limited”. A standardized dry hair lock is shaped in a curl on a cylinder with a diameter of 35 mm after having been coated with a styling composition, then wound on said cylinder and dried in place for a period of 24 hours. The curl is placed in the apparatus, which submits it to ten compression-decompression cycles along a vertical movement, and analyzes the compressive forces involved to calculate:
the longevity of the compressive strength: this is the complement to 100% of the absolute value of the relative variation of the maximum force before breakage of the film of styling product between the tenth cycle and the first cycle relative to the first cycle, expressed as a %; the greater the value, the greater the longevity of the strength,
the longevity of the compressive stiffness: this is the complement to 100% of the absolute value of the relative variation of the maximum force before breakage of the film of styling product between the tenth cycle and the first cycle relative to the first cycle, expressed as a %; the greater the value, the greater the longevity of the stiffness.

The measurement parameters used are: compression of 15%; 10 cycles; speed of 50 mm/min; contact force 5 gf; maximum force 2000 gf; gauging at each cycle. The Fibra Curl software uses the measurements to provide the values of the compressive properties (strength longevity, stiffness longevity). A “Wilcoxon Rank Sum” test was carried out to verify the statistically significant differences between the groups for each parameter. In all cases, a confidence level of 95% (p-value of 0.05) was reached.

Locks with a length of 18 cm and width 15 mm are prepared from a “European virgin ex IHIP” lock of hair. The locks are prepared according to the following protocol:

1/Washing

The lock is rinsed under tap water with a shower head in 35° C.+/−2° C. water at approximately 4 L/min for 30 seconds. The excess water is removed by gently pressing the lock between two fingers from the root to the tip three times. 1 mL of a solution of sodium laureth sulfate at 14% of dry matter is applied per 1 gram of wet hair, homogeneously on each lock. The lock is then manually pressed by a zigzag movement. The lock is then rinsed under the tap water at 35° C.+/−2° ° C. at approximately 4 L/min for 30 seconds. The excess water is removed by gently pressing the lock between two fingers from the root to the tip three times. The lock is left to dry overnight in an air-conditioned room at 20° C.

2/Application of the Styling Product

2 mL of styling solution to be tested is uniformly applied to a lock using a syringe. The lock is combed five times to distribute the solution over the entire lock. Each lock is clamped over its entire width to ensure that there is no space between the fibers of the lock. The lock is pulled between two gloved fingers three times to further distribute the styling solution and to reshape the lock to its maximum width. The lock is wound onto the round support with a diameter of 35 mm, and it is left to dry at 21° ° C.+/−2° C. for 24 hours. Once dry, the curled lock is carefully removed from the support.

3/Measurement

The curled lock is placed in the MTT175 apparatus configured for curl compression according to the manufacturer's recommendations. The automated measurement is started according to the parameters presented above.

The separately evaluated styling compositions are gels prepared according to the compositions of table 11.

TABLE 11

| | | | Styling gel with |
|---|---|---|---|
| | | Placebo | Beauté by |
| Phase | Ingredient | styling gel | Roquette ® ST 720 |
| A1 | Water | q.s. 100 | q.s. 100 |
| A1 | Beauté by Roquette ® ST 720 | 0 | 5 |
| A2 | Beauté by Roquette ® DS 112 | 5 | 5 |
| B | Beauté by Roquette ® PO 070 | 3 | 3 |
| B | Denat alcohol | 5 | 5 |
| C | Microcare PE | 1 | 1 |

TABLE 12

| | | With Beauté by |
|---|---|---|
| | Placebo | Roquette ® ST 720 |
| Longevity of compressive | 80.49 | 93.06 |
| strength | | |
| (%) | | |
| Longevity of compressive | 71.57 | 88.83 |
| stiffness | | |
| (%) | | |

For both the placebo styling gel and the styling gel with the pea starch Beauté by Roquette® ST 720, a decrease in the compressive strength and stiffness is observed when the curl is subjected to the cycle of ten compressions-decompressions. This decrease is advantageously lower for the styling cream with the pea starch Beauté by Roquette® ST 720.

The styling cream comprising the pea starch Beauté by Roquette® ST 720 results in styling with better compressive strength longevity, as well as better compressive stiffness longevity, than the placebo cream. The pea starch Beauté by Roquette® ST 720 makes it possible to increase both the compressive strength of the curl and the compressive stiffness of the curl.

The compositions according to the invention advantageously make it possible to increase the longevity of the compressive strength of the curl and/or for maintaining and preferentially increasing the longevity of the compressive stiffness of the curl.

### Example 3: Three-Point Bending

Here, the properties of bending of “flat strip”-shaped locks with a styling composition are evaluated without or with the pea starch Beauté by Roquette® ST 720. It is used according to the “3-point bend” measurement protocol of the “MTT175” apparatus from the manufacturer “Dia-Stron Limited”. A “flat strip” styled lock is subjected to a flexural stress at its center, while being held on supports at its ends. The data acquired by the apparatus are processed by the software “Fibra. Bending” which characterizes each tress by 4 parameters:
the maximum flexural strength: this is the maximum force to be applied to break the film of styling product on the lock,
the maximum flexural stiffness: this is the maximum of the force gradient to break the film of styling product,
the longevity of the flexural strength: this is the complement to 100% of the relative variation of the maximum force before breakage of the film of styling product between the tenth cycle and the first cycle relative to the first cycle, expressed as a %; the greater the value, the greater the longevity of the strength,
the longevity of the flexural stiffness: this is the complement to 100% of the relative variation of the maximum force before breakage of the film between the tenth cycle and the first cycle relative to the first cycle, expressed as a %; the greater the value, the greater the longevity of the stiffness.

The measurement parameters used for the maximum flexural strength and stiffness are: maximum deflection; 1 cycle; speed of 50 mm/min; contact force 5 gf; maximum force 2000 gf.

The measurement parameters used for the longevity of the flexural strength and stiffness are: deflection to be determined; 10 cycles; speed of 50 mm/min; contact force 5 gf; maximum force 2000 gf; gauging each cycle.

The locks of hair are identical to those used in example 2, and are prepared in the form of flat strip in a manner identical to example 2, following the protocol for washing and applying the product, with the difference that the final step of shaping is carried out by arranging the locks flat on a plate.

The separately evaluated styling compositions are gels prepared according to the compositions of table 13. They are in the form of gels.

TABLE 13

| | | Placebo | Styling gel with |
|---|---|---|---|
| | | styling | Beauté by |
| Phase | Ingredient | gel | Roquette ® ST 720 |
| A1 | Water | q.s. 100 | q.s. 100 |
| A1 | Beauté by Roquette ® ST 720 | 0 | 7 |
| A2 | Beauté by Roquette ® DS 112 | 5 | 5 |
| B | Beauté by Roquette ® PO 070 | 3 | 3 |
| B | Denat alcohol | 5 | 5 |
| C | Microcare PE | 1 | 1 |

TABLE 14

| | | With Beauté by |
|---|---|---|
| | Placebo | Roquette ® ST 720 |
| Maximum flexural strength | 602.52 | 894.81 |
| (in gf) | | |
| Maximum flexural stiffness | 147.20 | 100.57 |
| (in gf/mm) | | |
| Longevity of flexural strength | 91.93 | 98.63 |
| (%) | | |
| Longevity of flexural stiffness | 90.29 | 90.52 |
| (%) | | |

Regarding the maximum flexural strength, the styling composition with Beauté by Roquette® ST 720 provides a greater value than the placebo composition: this reflects the fact that Beauté by Roquette® ST 720 enables shaping with better strength than the placebo.

Regarding the maximum flexural stiffness, the styling composition with Beauté by Roquette® ST 720 provides a lower value than the placebo composition: this reflects the fact that Beauté by Roquette® ST 720 enables shaping with lower stiffness than the placebo, and therefore a more flexible shaping than the placebo, and thus a lower “cardboard” effect compared to the placebo.

Regarding the longevity of the flexural strength, the styling composition with Beauté by Roquette® ST 720 provides a value of the percentage of change closer to zero, and lower in absolute value than the placebo: this reflects the fact that Beauté by Roquette® ST 720 allows a shaping with better flexural strength/resistance, and better resistance to mechanical stresses.

Regarding the longevity of the flexural stiffness, the styling composition with the pea starch Beauté by Roquette® ST 720 provides a value of percentage of change virtually equal to the placebo: this reflects the fact that the pea starch Beauté by Roquette® ST 720 allows a stiffness equivalent to the placebo, and therefore an equivalent flexural resistance stiffness.

Thus, the compositions according to the invention further make it possible to increase the maximum flexural strength, reduce the maximum flexural stiffness, to increase the longevity of the flexural strength of a curl and to increase the longevity of the flexural stiffness of a curl.

The pea starch Beauté by Roquette® ST 720 therefore makes it possible to increase the styling/fixing power, while reducing the “cardboard” effect, and increasing the longevity of the curls/hairstyle.

### Example 4: Thermal Protection

Here, the thermal protection property of the hair is evaluated by the pea starch Beauté by Roquette® ST 720 through an ex-vivo test for quantifying the oxidation of the hair proteins under the action of heat. Quantification of the oxidation is carried out by epifluorescence microscope imaging associated with densitometric analysis of the images. The quantifications are made in two views: the longitudinal view (that is, view of the hair in its length), and the sagittal view (that is, view of the section of the hair after cutting). The longitudinal view makes it possible to quantify the oxidation on the surface of the hair over a portion of its length. The sagittal view makes it possible to quantify the oxidation in two parts of the hair: the cuticle (that is, the fine outer layer) and the cortex (that is, the body of the hair).

Three groups of locks consist of:
a “control” group, that is, a negative control: these locks are not treated, and are kept at room temperature throughout the duration of the test,
a “positive control” group: these locks are not treated, and are exposed to 95° C. heat for 20 minutes,
a group treated with an aqueous pea starch solution Beauté by Roquette® ST 720, and exposed to 95° C. heat for 20 minutes.

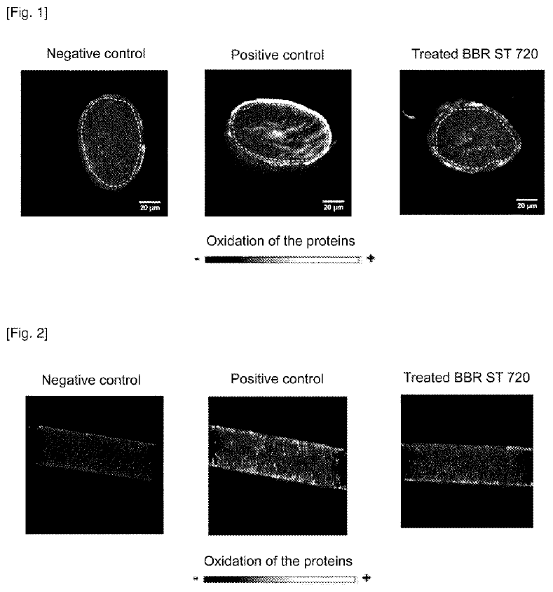

Three hairs are taken and analyzed by group of locks, and the oxidation level of the group is calculated. This oxidation level of the group is the average of the fluorescence intensities of the group normalized by the average intensity of the sample of the negative control group. FIGS. 1 and 2 show the microscopic fluorescence images according to the longitudinal view and according to the sagittal view respectively.

The thermal oxidation protection property for the treated group is calculated according to the formula:

Protection (%)=[Oxidation Level(Positive Control)−Oxidation Level(Treated Group)]/[Oxidation Level(Positive Control)−Oxidation Level(Negative Control)]×100

In table 15, the oxidation level (normalized to the negative control) is listed.

TABLE 15

| | Negative | Pos. | With BBR |
|---|---|---|---|
| | control | Control | ST720 |
| Sagittal view: all | 100 | 194 | 144 |
| Sagittal view: cuticle | 100 | 239 | 156 |
| Sagittal view: cortex | 100 | 182 | 141 |

Table 16 shows the results relating to oxidation protection.

TABLE 16

| | Negative | Pos. | With BBR |
|---|---|---|---|
| | control | Control | ST720 |
| Sagittal view: all | 100% | 0% | 54% |
| Sagittal view: cuticle | 100% | 0% | 59% |
| Sagittal view: cortex | 100% | 0% | 50% |

The densitometric analysis of the fluorescence measured on the entire sagittal view of the hair treated with the pea starch Beauté by Roquette® ST 720 and subjected to heating, shows that the oxidation of the proteins is significantly lower than for the positive control. This reduction in protein oxidation is measured both in the cuticle and in the cortex. The pea starch Beauté by Roquette® ST 720 thus protects the hair from heat-induced oxidation.

The leguminous-plant starch advantageously makes it possible to protect the hair from stress and therefore to prevent and reduce hair stress.

1. A non-therapeutic use of a modified leguminous-plant starch to increase the retention of hair curls and/or to maintain and preferentially increase the longevity of the compressive strength of the curl and/or to maintain and preferentially increase the longevity of the compressive stiffness of the curl.

2. The non-therapeutic use according to claim 1, wherein the modified leguminous-plant starch makes it possible to increase curl length retention and/or curl tightness retention.

3. The non-therapeutic use according to claim 1, wherein the modified leguminous-plant starch further makes it possible to increase the maximum flexural strength and/or reduce the maximum flexural stiffness and/or increase the longevity of the flexural strength of a curl and/or to increase the longevity of the flexural stiffness of a curl.

4. A non-therapeutic use of a modified leguminous-plant starch to prevent, eliminate or reduce hair stress.

5. The non-therapeutic use according to claim 4, wherein the modified leguminous-plant starch makes it possible to prevent, eliminate or reduce hair oxidation induced by thermal stress and/or chemical stress.

6. The non-therapeutic use according to claim 1, wherein the leguminous-plant starch is a pea starch.

7. The non-therapeutic use according to claim 1, wherein the starch is a hydrolyzed and hydroxyalkyl starch, preferably a hydroxypropylated starch.

8. A cosmetic composition in the form of an oil-in-water emulsion comprising:
at least one leguminous-plant starch,
at least one volatile oil,
at least one non-volatile oil,
at least one wax,
at least one surfactant,
water

9. A cosmetic composition in the form of an oil-in-water emulsion comprising:
at least one leguminous-plant starch,
at least one volatile oil,
at least one non-volatile oil,
at least one wax,
at least one surfactant,
water,
at least one gelling agent,
at least one colorant.

10. A method of shaping hair curls and/or of preventing, removing or reducing hair stress, comprising a step of applying a composition according to claim 8 to the hair.

11. The non-therapeutic use of a cosmetic composition according to claim 8, for shaping hair, preferentially shaping hair curls.

12. The non-therapeutic use according to claim 11, to increase hair curl retention and/or to maintain and preferentially increase the longevity of the strength of the curl and/or to maintain and preferentially increase the longevity of the stiffness of the curl.

13. The non-therapeutic use according to claim 11, wherein the leguminous-plant starch further makes it possible to increase the maximum flexural strength and/or reduce the maximum flexural stiffness and/or increase the longevity of the flexural strength of a curl and/or to increase the longevity of the flexural stiffness of a curl.

14. The non-therapeutic use of a cosmetic composition according to claim 8, to prevent, eliminate or reduce hair stress.

15. The non-therapeutic use according to claim 14, wherein the cosmetic composition makes it possible to prevent, eliminate or reduce hair oxidation induced by thermal stress and/or chemical stress.

16. A method of shaping hair curls and/or of preventing, removing or reducing hair stress, comprising a step of applying a composition according to claim 9 to the hair.

17. The non-therapeutic use of a cosmetic composition according to claim 9, for shaping hair, preferentially shaping hair curls.
